(19) United States
(12) Patent Application Publication
(10) Pub. No.: US 2018/0092696 A1
(43) Pub. Date: Apr. 5, 2018

(54) CONTEXTUAL CREATION OF REPORT CONTENT FOR RADIOLOGY REPORTING

(71) Applicant: KONINKLIJKE PHILIPS N.V., EINDHOVEN (NL)

(72) Inventors: YUECHEN QIAN, LEXINGTON, MA (US); JOOST FREDERIK PETERS, ULTRECHT (NL); JOHANNES BUURMAN, 's-Hertogenbosch (NL); VLADO KOZOMARA, NASHVILLE, TN (US); KEVIN MCENERY, EINDHOVEN (NL)

(21) Appl. No.: 15/548,785

(22) PCT Filed: Jan. 28, 2016
(86) PCT No.: PCT/IB2016/050422

§ 371 Date: Aug. 4, 2017

Related U.S. Application Data
(60) Provisional application No. 62/112,183, filed on Feb. 5, 2015.

(51) Int. Cl. G06F 19/00; G06F 3/16; G06F 17/27

(52) U.S. Cl. CPC A61B 34/10; G06F 17/2785; G06F 3/167; G06F 19/00

(57) ABSTRACT

A medical diagnostic reporting system monitors a diagnostician's activities performed on medical images while developing a diagnosis, extracts image context and relevant data based on these activities, then transforms the relevant data into a structured narrative based on the image context. The structured narrative is presented to the diagnostician in a non-intrusive manner, and allows the diagnostician to select whether to insert the structured narrative into the ongoing diagnostic report.

### FIELD OF THE INVENTION

This invention relates to the field of medical diagnostic systems, and in particular to a medical diagnostic system that facilitates automation of diagnostic reports by transforming data from a medical imaging system into a structured/templated narrative for inclusion in a diagnostic report.

### BACKGROUND OF THE INVENTION

A substantial portion of a medical diagnostician's time is consumed by the need to create a diagnostic report. The report must include administrative information, such as an identification of the patient, the patient's condition, and the tests conducted, as well as the results obtained, the specific findings, and the determined prognosis.

Conventionally, the diagnostician types or dictates the diagnostic report while accessing the medical images upon which the diagnosis is based. The diagnostician may identify a region of interest in the image, such as a particular organ, then identify abnormalities, such as lesions, within the region of interest. The diagnostician will typically use a medical imaging system to measure relevant parameters, such as the size and/or volume of the abnormality, the location of the abnormality, and so on. Depending upon the diagnostician's preferences, the diagnostician may take note of these parameters, then use these notes afterward while composing the diagnostic report; or, the diagnostician may have a speech-recognition system operating concurrently with the diagnostic system, and may dictate the diagnostic report ‘on-the-fly’ while performing the diagnostic measurements.

In some cases, the diagnostic report is created solely for the diagnostician's records, but in a number of fields, such as radiology, the diagnostician's report is intended to be communicated to another party, such as the patient's doctor or surgeon, and must conform to accepted standards.

DICOM (Digital Imaging and Communications in Medicine) is a standard for storing, printing, and communicating medical imaging information that enables the integration of imaging and networking hardware from multiple manufactures into a Picture Archiving and Communication System (PACs) that networks computers used at labs, hospitals, doctor's offices, and so on. PACS enables remote access to high-quality radiologic images, including conventional films, CT, MRI, PET scans and other medical images over the network.

At the application level (“layer 7” in the OSI model), Health Level-7 or HL7 includes a set of international standards for transfer of clinical and administrative data between hospital information systems. HL7 develops conceptual standards (e.g., HL7 RIM), document standards (e.g., HL7 CDA), application standards (e.g., HL7 CCOW), and messaging standards (e.g., HL7 v2.x and v3.0).

In a diagnostic recording system, some information, such as the aforementioned administrative information, may be transferred by command from the medical imaging system to the diagnostic report. Other data elements, however, such as comparison, image references, measurements, and follow-up recommendations, must be entered by the user (typed, dictated, etc.), which is both time-consuming and error-prone.

Also, the descriptive text is narrative by nature and may differ from person to person. In a voice-recognition system, these differences increase the difficulty for natural language processing or other computer techniques to analyse the text, and the diagnostician's time is spent examining the text inserted by the voice-recognition system. Even in a non-voice-recognition system, the use of different narratives in describing a finding may occasionally introduce confusion, or even mis-interpretation, by the recipient.

### SUMMARY OF THE INVENTION

It would be advantageous to provide a system and process that facilitates the transfer of relevant information from a medical imaging system for inclusion in a diagnostic report. It would also be advantageous to transform the relevant information into a standard form for inclusion in the report.

To better address one or more of these concerns, in an embodiment of this invention, the medical diagnostic reporting system monitors a diagnostician's activities performed on medical images while developing a diagnosis, extracts image context and relevant data based on these activities, then transforms the relevant data into a structured narrative based on the image context. The structured narrative is presented to the diagnostician in a non-intrusive manner, and allows the diagnostician to select whether to insert the structured narrative into the ongoing diagnostic report. Alternatively, the structured narrative is used to populate the machine clipboard, in anticipation of the diagnostician including it in the report immediately. As the diagnosis continues, additional relevant information is transformed into additional structured narratives for optional insertion into the diagnostic report. If the user does not choose to insert a particular structured narrative within a given time duration, that structured narrative may be deleted; otherwise the structured narrative can be archived and retrieved for later use.

The system may use a predefined vocabulary or a semantic ontology-based matching process to transform the relevant data into the structured narrative. In some embodiments, the diagnostician is given the option of identifying images and/or regions of interest in an image to extract the image context and relevant information.

The system may be further implemented via automatic data transfer. The diagnostic viewing system provides application programming interfaces (API) that can retrieve the structured narrative text from the viewing system. The reporting system, which can come from a vendor different from that of the diagnostic viewing system, can retrieve and insert the structured narrative text automatically, by invoking the API provided by the diagnostic viewing system.

### BRIEF DESCRIPTION OF THE DRAWINGS

The invention is explained in further detail, and by way of example, with reference to the accompanying drawings wherein:

FIG. 1 illustrates an example flow diagram for automating the transfer of information derived from medical images to a diagnostic report.

FIG. 2 illustrates example structured narrative skeletons.

FIG. 3A illustrates an example display of selectable structured narrative elements.

FIG. 3B illustrates an example diagnostic report based on a selection of elements of FIG. 3A.

FIG. 4 illustrates an example user interface that facilitates the transfer of information derived from medical images to a diagnostic report.

FIG. 5 illustrates an example block diagram of a medical diagnosis system that facilitates the transfer of information derived from medical images to a diagnostic report.

Throughout the drawings, the same reference numerals indicate similar or corresponding features or functions. The drawings are included for illustrative purposes and are not intended to limit the scope of the invention.

### DETAILED DESCRIPTION

In the following description, for purposes of explanation rather than limitation, specific details are set forth such as the particular architecture, interfaces, techniques, etc., in order to provide a thorough understanding of the concepts of the invention. However, it will be apparent to those skilled in the art that the present invention may be practiced in other embodiments, which depart from these specific details. In like manner, the text of this description is directed to the example embodiments as illustrated in the Figures, and is not intended to limit the claimed invention beyond the limits expressly included in the claims. For purposes of simplicity and clarity, detailed descriptions of well-known devices, circuits, and methods are omitted so as not to obscure the description of the present invention with unnecessary detail.

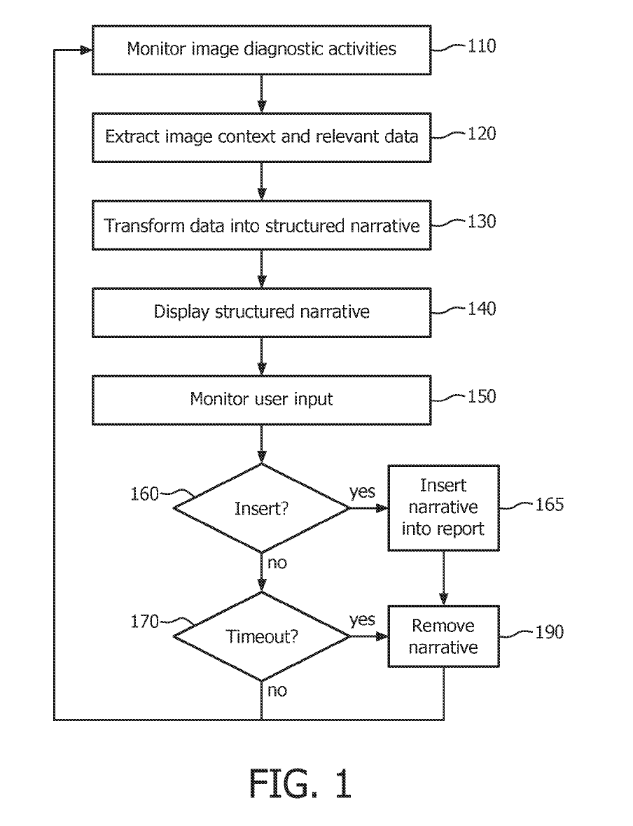

FIG. 1 illustrates an example flow diagram for automating the transfer of information derived from medical images to a diagnostic report.

At 110, a diagnostician's activities are monitored/recorded while the diagnostician (user) is performing diagnoses of medical images. The user may be, for example, a radiologist who may be reviewing images of a patient obtained from a CT-scan, an MRI, an X-ray, and so on, to identify abnormalities, or to confirm the absence of abnormalities. In some cases, the radiologist may be reviewing a series of images of the patient taken over time, to compare these images and identify changes over time.

One of skill in the art will recognize that any of a variety of techniques, or combinations of techniques may be used to identify the individual tasks that the user is performing at any given time.

In an embodiment of this invention, the monitoring may be performed while the diagnostician is using a conventional medical diagnostic system or tool, and the user's keystrokes, mouse clicks, gaze points, gestures, voice commands, and so on, are monitored and processed in the background to identify each particular task that is being performed (pan, zoom, select, measure, group, highlight, and so on), based on the user's actions.

In other embodiments, the medical diagnostic system or tool may be modified to ‘trace’ the flow of the diagnosis by identifying which particular sub-routines are being invoked, and in what order. To reduce the complexity of the tracing, the higher-level routines that are invoked to perform a given task are predefined, and only the invocation of these routines are recorded.

Based on the monitored actions, the particular diagnostic tool being used, the particular organ being diagnosed, the particular modality of the images, and so on, the context of the diagnosis may be determined, at 120. For example, the context may be one of: identifying the patient, body part, symptoms, etc.; identifying, annotating and/or measuring elements, such as lesions; comparing images of an organ at different times; selecting and identifying images to support the findings; and so on.

Within each context, certain parameters may be defined as being relevant to the task. In the context of initially opening a patient's file, for example, the reporting system may anticipate that the diagnostic report is likely to include such data as the patient's name, the patient's medical profile, the current date, the diagnostician's name. When a particular image set is accessed, the system may anticipate that an identification of the body part, the image set, and the date the image set was created would likely to be included in the diagnostic report. In an example embodiment, the system may anticipate/predict the context and/or the relevant data based on one or more models of sequences typically performed during the diagnostic process. Different models may be provided for different types of diagnosis, different types of image modality, different diagnosticians, and so on.

In the context of dealing with lesions, the location and size (extent, area, and/or volume) are generally relevant parameters, as may be shape (oval, bullseye, etc.), composition (fluid, hardened, etc.), characterization (benign, malignant, etc.), and so on. In the context of time-differing images, other parameters may be relevant, including the date of each image. The relevant parameters may also be dependent upon the particular body part being examined and other factors. The values of the relevant parameters are extracted from the medical diagnostic system or tool as they are determined during the diagnostic process, at 120.

At 130, the extracted relevant information is transformed into a structured narrative, the form of which may be based on the extracted context. The structured narrative may be created based on a set of predefined statements or ‘skeletons’ within each context, into which the relevant parameters are inserted.

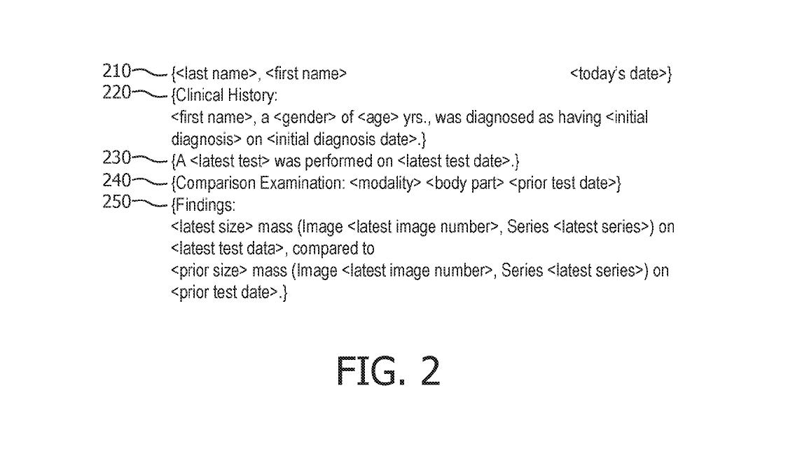

FIG. 2 illustrates a set of example structure narrative skeletons 210-260, each skeleton enclosed by brackets (( )). Skeleton 210 includes parameters <last name>, <first name>, <today's date> and may be accessed and filled in with the current patient's name and the current date when the patient's record is first accessed. At that time, skeleton 220 may be accessed and filled in, using the patient's gender, age, and initial diagnosis.

When the diagnostician accesses a particular record in the patient's file, such as the latest test images, skeleton 230 may be filled in with the name of the test and the test date. Optionally this skeleton 230 may be filled in as information that is likely to be included in a report, regardless of whether the diagnostician is accessing that particular test.

Skeleton 240 may be accessed and filled in when the system detects that the diagnostician has accessed images or results of a prior test. As the diagnostician (or the diagnosis system) identifies corresponding features in the current and prior test images, skeleton 250 may be accessed and filled in to provide the current and prior size of the identified feature.

One of skill in the art will recognize that the skeletons of FIG. 2 are merely examples presented for illustrative purposes, and that any of a variety of forms may be used. For example, in the case of comparing images taken at different times, an introductory structured narrative could be of the form:

“(<latest date>, <body part>, <modality>):(<prior date>, <body part>, <modality>)” where the date of the most recent test would be inserted for <latest date>, the body part (e.g. “abdomen”, “right lung”, etc.) inserted for <body part>, and the modality (e.g. “CT”, “MRI”, etc.) for the <modality>. In like manner the appropriate insertions would be made for the prior test. The “:” symbol may be defined to signify “one or more”, so that the information from more than one prior test can be inserted using a repeat of the given format.

In the identification of a particular element type, such as a lesion, the structured narrative may be of the form:
“<type>, [<body part>,]<location>, <units>, <size1>: <sizeN>”.
   Depending upon the particular context, the <location> field may be provided in the form of coordinates, as an identifier of an anatomical location, a general location (“upper left”), and so on. In like manner, the <units> may serve to identify whether the measured sizes refer to a length, an area, a volume, an angle, and so on. In this example the brackets “[” “]” identify that the <body part> field is optional, depending upon whether the body part has already been unambiguously identified.

The structured narrative may also identify the particular characteristics of the image from which the information is based:
“<body part>, <modality>, <view direction>, [<magnification>]”.

In like manner, the structured narrative may include a reference to the current image:
“[<date-time>,]<series#>, <image#>[: <imageN#>], <modality>, <body part>”.

It should be noted that the particular form of the structured narrative may be dependent upon the target recipient, or the target medium. If the target recipient, for example, is the patient, the above introductory structured narrative might be in a more “patient readable” form, such as:
“This diagnosis is based on the results of the <modality> images of your <body part> obtained on <latest date>, as compared to the results of the <modality> images of your <body part> obtained on <prior date>.”

The structured narrative may also conform to a particular standard, such as DICOM, ML7, and so on.

It should also be noted that different forms of the structured narrative may be provided using the same relevant information. That is, a terse form of the structured narrative may be presented to the diagnostician for potential selection, as detailed further below, but a longer form of the structured narrative may be inserted into the actual diagnostic report. In like manner, multiple diagnostic reports may concurrently be created: one for a medical practitioner, and one for the patient.

For the purposes of this disclosure, a “structured narrative” is merely an organization of relevant data in a form that is consistent regardless of the particular diagnostician, and regardless of the particular patient. That is, if two different diagnosticians create a ‘patient readable’ diagnostic report for different patients, the form of the report with regard to the relevant information will be the same. In some embodiments, the user is able to define the form of the structured narrative; in such an embodiment, once the structured narrative is created, the output will be consistent for all subsequent users of this new structured narrative.

At 140, the structured narrative is presented to the diagnostician for the diagnostician's consideration for inclusion in the diagnostic report. In an example embodiment, this structured narrative is presented in an unobtrusive manner, such as in a window that appears in a corner of the diagnostic system display, or on an adjacent display. Generally, this structured narrative will contain the relevant data in a terse form, because the diagnostician is aware of the current context, and needs minimal additional information.

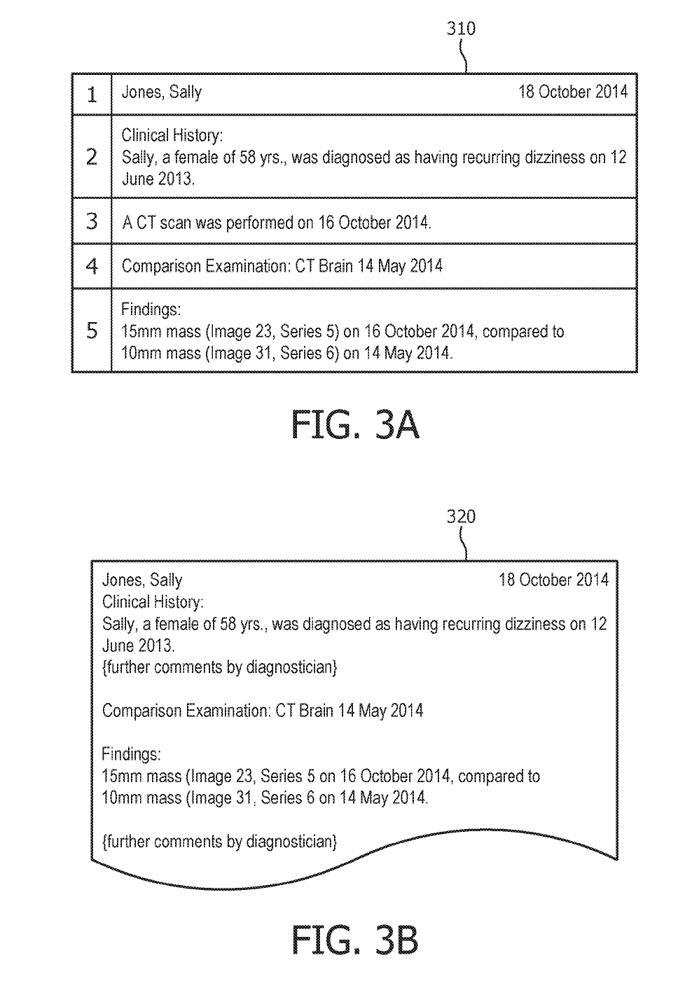

FIG. 3A illustrates an example presentation of structured narratives for a diagnostician's selection, based on the diagnostician's actions during the current session, using the example skeletons of FIG. 2.

When the diagnostician initially accesses a patient's record, skeletons 210, 220, 230 may be accessed and filled in with this patient's information to provide selectable elements 1, 2, and 3. As the diagnostician proceeds to access image information to perform the diagnosis, the system may access skeletons 240, 250, to provide selectable elements 4 and 5 of FIG. 3A.

The user's input in monitored, at 150, to determine whether the user wants the structured narrative to be inserted into the diagnostic report. As also noted above, depending upon the diagnostician's preferences, the selected narratives may be placed in a ‘notebook’ that is subsequently edited by the diagnostician to add text that couples and further explains the individual selected narratives. Alternatively, the diagnostician may prefer to create the diagnostic report ‘on-the-fly’ using, for example, a speech-recognition system that captures the diagnostician's spoken words and directly inserts the structured narrative each time the diagnostician indicates that the selected narrative should be inserted.

In an example system, the user may voice a command, such as “Insert that”, or, if multiple structured narratives have been presented to the user, the user may say “Insert number three”, or “Insert lesion details”. One of skill in the art will recognize that any of a variety of techniques may be used to identify the structured narrative that is to be inserted, including for example, via keyboard, mouse, touch pad, touch screen, and so on, as well as gesture recognition, gaze tracking, and so on.

If, at 160 of FIG. 1, the user selects an item to be inserted, the structured narrative is placed in the diagnostic report, at 165. The example diagnostic report 320 of FIG. 3B illustrates the results of a diagnostician selecting all of the elements of FIG. 3A except element 3 (skeleton 230).

Upon selection, the selected narrative may be removed from the options presented to the user, at 190. As noted above, the form of the structured narrative that is inserted may differ from the form of the structured narrative that is displayed for the user's selection, but the relevant information will be the same.

If, at 160, the user does not choose to insert the structured narrative, the time that each narrative has been made available for selection is determined, and if a narrative has been available but not selected exceeds a given time limit, at 170, it is removed from the selectable elements, at 180. In lieu of a time limit, the number of structured narratives presented to the user at one time may be limited, and the oldest structured narrative is deleted each time this limit is reached. Depending upon the particular embodiment, and/or the particular user's preferences, the removed structured narratives may be archived for subsequent use, or they may be deleted.

The system continues to monitor the user's diagnostic activity and generate structured narratives for optional insertion into the diagnostic report, as indicated by the loop back to block 110. In this manner, the user is relieved of having to transcribe the relevant information into the diagnostic report, and the recipient of the diagnostic report receives the relevant information in a well structured form, thereby minimizing errors and/or mis-interpretations.

Although the above example of selectable narratives of FIG. 3A is illustrative of a reporting system that displays selectable narratives independent of the diagnosis system, one of skill in the art will recognize that the selection process may be integral to the diagnosis system.

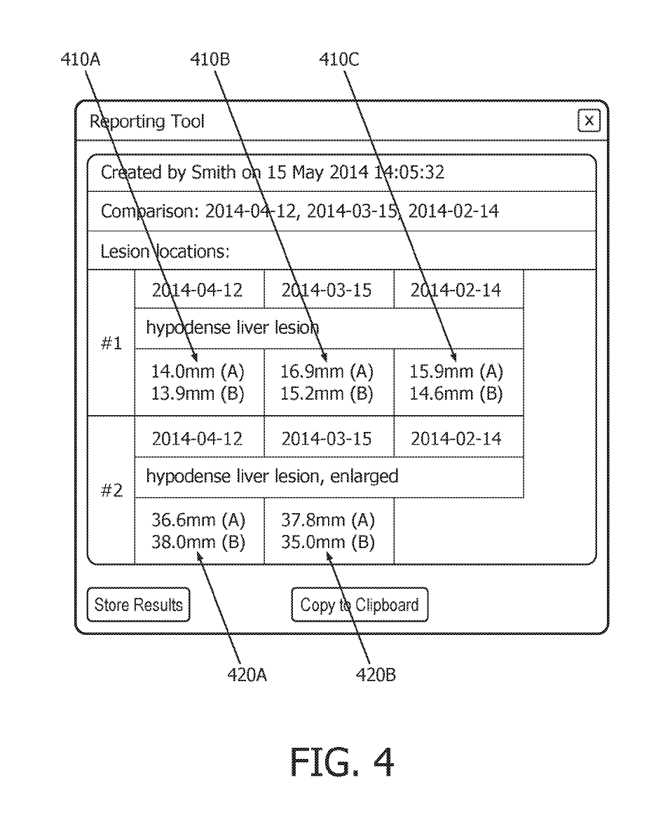

FIG. 4 illustrates an example user interface that facilitates the transfer of information derived from a medical diagnosis system to a diagnostic report system. In this example, the dimensions of a lesion at different times are reported by the diagnosis system, and the user is given the option of selecting which information items 410A-C, 420A-B are to be inserted in the diagnostic report. In a straightforward embodiment, the user may use a mouse to select one or more of the reports, then click on the “insert” key 450. In a voice-recognition system, the user may say “Insert number one”, which would insert the three reports 410A-C, or “Insert latest sizes”, which would insert reports 410A and 420A. In a gaze tracking embodiment, the user may gaze at a report, then double-blink to have it inserted in the report.

Depending upon the particular embodiment, the selected displayed information may be copied directly into the diagnostic report, or is processed to conform to identified skeletal forms.

To facilitate such integration, particularly in configurations where different vendors provide different components, the diagnosis system may include application programming interfaces (APIs) that can be structured to export information being displayed to external systems, and the reporting system may use these APIs to retrieve the information from the viewing system. In some embodiments, the APIs may be configured to provide the information directly, or to provide the information in a structured narrative form. That is, the processes of this invention may be distributed among multiple physical systems.

In an example embodiment, the APIs may be configured to provide the parameters directly, such as via a call such as “Get (body_part, modality, date)”, which will return the current value of these parameters at the diagnosis system. In another embodiment, wherein the diagnosis system is configured to provide structured narratives, the call may be of the form “Get (Finding)”, which will return a structured narrative such as produced by skeleton 250 of FIG. 2 (selectable element 5 in FIG. 3A).

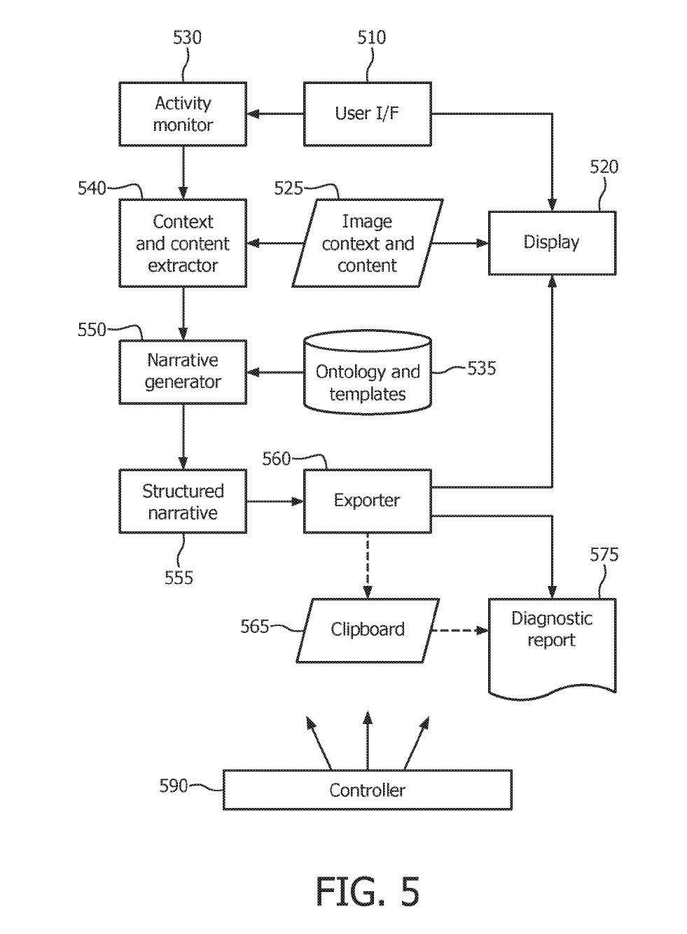

FIG. 5 illustrates an example block diagram of a diagnosis reporting system that facilitates the transfer of information derived from medical images to a diagnostic report. The diagnostic reporting system of FIG. 5 is presented in the context of a radiologist using a diagnostic image viewing system.

In this example embodiment, the radiologist interacts with the diagnostic image viewing system via a user interface 510, and the structured narratives that are determined during the diagnostic process are presented to the radiologist on a display 520, which may be part of the diagnostic image viewing system. A controller 590 manages the interactions among the elements in the diagnostic reporting system; for ease of illustration, the connections between the controller 590 and each of the other elements in FIG. 5 are not illustrated.

An activity monitor 530 constantly monitors activities performed by the diagnostician in the diagnostic image viewing system, including mouse clicks/keystrokes, opening/closing of studies, scrolling/viewing prior studies, linking images, measuring/annotating lesions, searching relevant images and recommendations, and so on.

A context and content extractor 540 assesses the interactions and the output provided by the diagnostic image viewing system to determine the current diagnosis context and extract the relevant data associated with the completed task. The extractor 540 may access the medical images 525 directly to facilitate the context determination and data extraction, or it may access the output of the diagnosis image viewing system, or a combination of both.

The extractor 540 may perform different assessments depending upon the current context. For example, when the radiologist is loading or closing a study, the extractor 540 may determine what studies are used as baseline. The radiologist's actions of scrolling, viewing, or enlarging prior studies and/or the linking of current and prior images facilitate identifying which prior studies are actually used, thereby establishing the baseline. In this case, the system automatically captures the date, time, modality, body part (including study accessions) of each of the studies.

When the radiologist is measuring or annotating a lesion, the extractor 540 may detect the current finding of interest and automatically capture the image/series information, date/time, body part, and modality of the study in which a finding is annotated or measured. For example, the extractor 540 may capture:
the XY location and text of an annotation;
the XY location, length/size/volume/angle (whenever applicable) of the finding;
the anatomical location, body part, laterality associated with the finding, with help of imaging processing algorithms or anatomy region approximation algorithms (using Z-index);
the view of the image (axial/sagittal/coronal) from DICOM meta demo;
the current window width/level of the finding;
whether two/multiple measurements intersect, and if so, merge them into a single finding; and
the current image as a key image, including image/series information, the date, time, modality, body part of the study (including Image UID, Series UID), and the current window width/level of the image.

Depending upon the level of interaction provided between the extractor 540 and the diagnostic image viewing system, the extractor 540 may use a variety of techniques to extract the context and content information. For example, if the diagnostic image viewing system can be configured to send HL7 messages, the extractor 540 may be configured to receive/absorb HL7 feeds. If the diagnostic image viewing system provides an API (Application Program Interface) for accessing information, the extractor 540 may be configured to send queries to the API for the context and content information. In some embodiment, the extractor 540 may be configured to enable the radiologist to copy relevant information into a ‘clipboard’, then transfer the relevant information to the extractor 540 via a ‘paste’ command. If the copied information is captured as an image from the image viewing system, the extractor 540 may include a text-recognition element that extracts the information from the copied image.

The narrator generator 550 uses the extracted information to generate the structured narrative 535, by providing a templated/formatted description of the current action and its context. As detailed above, the description of the current action and its context may use predefined templates to maintain consistency across users and enables easy parsing of the reports using natural language processing. An ontology and template database 535 facilitates this creation of the structured narrative 555.

An exporter 560 receives the radiologist's selections via the user interface 510 and selectively copies and pastes the generated narrative into the diagnostic report. The exporter 560 also checks the validity of an action and the context and updates the system memory accordingly. If the action was performed but the generated description was not consumed, it invalidates the generated description and cleans up its memory to avoid potential data synchronization errors.

The exporter 560 may effect the transfer of the structured narrative 535 in a variety of ways, as detailed above, including voice commands, mouse clicks, gestures, and so on. In some embodiments, the exporter 560 uses the ‘clipboard’ that is provided in most operating systems to receive/copy the selected structured narrative, and pastes the structured narrative into the diagnostic report by interacting with a conventional word processor.

While the invention has been illustrated and described in detail in the drawings and foregoing description, such illustration and description are to be considered illustrative or exemplary and not restrictive; the invention is not limited to the disclosed embodiments.

For example, although the invention is presented in the context of a highly interactive process, it is possible to operate the invention in an embodiment wherein the process occurs in the background while each diagnosis is being performed, without any involvement by the diagnostician. The output report may be a text file that may be edited by the diagnostician after the diagnosis is completed. Alternatively, it may be a text file that documents the diagnostic process, including actions of the diagnostician, automated actions of the diagnosis system, the results of these actions, and so on.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A non-transitory computer-readable medium that includes a program that, when executed by a processor, causes the processor to:
monitor activities of a user that are performed on medical images during diagnostic viewings to determine a context via detection of a predetermined activity pattern;
extract predetermined data from the medical images and/or from the viewing settings of the images according to the determined context as relevant data;
transform the relevant data into one or more structured narratives by inserting the relevant data into one or more respective predefined skeletons;
provide a user with an option to insert at least one of the one or more structured narratives into a diagnostic report;
if the user selects to insert the at least one structured narrative, modify the diagnostic report to include the structured narrative.

2. (canceled)

3. The medium of claim 1, wherein the program causes the processor to: store each of the plurality of structured narratives in a memory, and enable the user to retrieve the plurality of structured narratives at a time different from a time that the activities are monitored.

4. The medium of claim 1, wherein the program causes the processor to use a semantic ontology-based matching process to transform the relevant data into the one or more structured narrative.

5. The medium of claim 1, wherein the program causes the processor to use a predefined vocabulary to transform the relevant data into the one or more structured narrative.

6. The medium of claim 1, wherein the program causes the processor to determine the relevant data by enabling the user to indicate a region of interest on one or more of the medical images.

7. The medium of claim 1, wherein the program causes the processor to determine the context by enabling the user to select one or more of the medical images.

8. The medium of claim 1, wherein the program causes the processor to use voice-recognition to enable the user to indicate the option to insert the one or more structured narrative into the diagnostic report.

9. The medium of claim 1, wherein the program causes the processor to remove the option of selecting the one or more structured narrative after a given duration since creation of the structured narrative.

10. (canceled)

11. The medium of claim 1, wherein the program causes the processor to: obtain patient identification information as the relevant data when the user first accesses a record of the patient, and transform the patient identification information as a first structured narrative for insertion in a newly created diagnostic report.

12. The medium of claim 1, wherein the program causes the processor to predict a next context based on a model of diagnostic sequences.

13. A diagnostic reporting system comprising:
a source of medical images associated with a patient;
an activity monitor that monitors a user's activities while accessing the medical images;
a context extractor that determines a context based on the user's activities via detection of a predetermined activity pattern;
a data extractor that extracts predetermined data from the medical images and/or from the viewing settings of the images according to the determined context as relevant data;
a narrative generator that transforms the relevant data into one or more structured narratives by inserting the relevant data into one or more respective predefined skeletons;
a user interface that enables a user to select the at least one of the one or more structured narratives for inclusion in a diagnostic report; and
an exporter that inserts the at least one structured narrative into the diagnostic report, if the user selects the at least one structured narrative for insertion.

14. (canceled)

15. The system of claim 1, wherein:
the extractor stores each of the plurality of structured narratives in a memory, and
the user interface enables the user to retrieve the plurality of structured narratives at a time different from a time that the activities are monitored.
